(19) United States
(12) Patent Application Publication
(10) Pub. No.: US 2004/0146919 A1
(43) Pub. Date: Jul. 29, 2004

(54) Method for early prediction of the onset of invasive cancer

(76) Inventor: Douglas D. Burkett, Gilbert, AZ (US)

Correspondence Address: William H. Drummond, DRUMMOND & DUCKWORTH, Suite 440 East Tower, 5000 Birch Tower, Newport Beach, CA, 92660, US

(21) Appl. No.: 10/758,936

(22) Filed: Jan. 14, 2004

Related U.S. Application Data
(63) Continuation of application No. 10/111,545, filed on May 18, 2002, now abandoned.
Application No. 10/111,545 is a 371 of international application No. PCT/US00/26551, filed on Sep. 26, 2000.

(51) Int. Cl. C12Q 1/68; G01N 33/48

(52) U.S. Cl. 435/6; 435/40.5

(57) ABSTRACT

A prognostic method for early prediction of eventual development of invasive cancer comprises identifying suspect cancerous and precancerous tissue sites by applying a dye which selectively stains such sites, followed by molecular genetic analysis to determine whether the DNA of such sites exhibits allelic losses for mutation of tumor suppressor genes.

This application is a Continuation of U.S.A. patent application Ser. No. 10/111,545, filed Sep. 26, 2000.

This invention relates to a prognostic method for early prediction of eventual development of invasive cancer.

### BACKGROUND OF THE INVENTION

Identification of Precancerous

Tissue by Genetic Analysis

All neoplasms are believed to arise from cells that have undergone genetic alteration, followed by clonal expansion. These genetic alterations include activation of proto oncogenes and inactivation of tumor suppressor genes. Identification of specific genetic changes with pathologic progression has allowed delineation of molecular progression models many tumors, including oral cancers. These progressions models provide molecular markers to aid in the diagnosis and detection of human tumors.

Molecular analysis has revolutionized the ability to look at specific genetic changes in primary human neoplasms. Powerful PCR-based techniques allow the use of routinely prepared tissue specimens embedded in paraffin for DNA extraction and subsequent genetic analysis. DNA extracted from these sample can be amplified to reveal various genetic alterations, including allelic losses. Such allelic losses (chromosomal deletions) are markers for the inactivation of critical tumor suppressor genes contained within the region of loss.

In adult tumors, tumor suppressor genes require a two-step inactivation process, whereby both parental allele must be knocked out for tumor progression. Generally, point mutation of one allele is followed by deletion of the second allele and complete loss of tumor suppressor function. These allelic losses deletions identified by microsatellite analysis represent a common second inactivation step for tumor suppressor gene inactivation. In practice, allelic loss (or deletions) can be determined by using highly polymorphic markers able to distinguish the maternal and paternal alleles in normal DNA. This pattern can then be compared to tumor DNA where recombination or deletion is represented as loss of either the maternal or paternal allele (also known as loss of heterozygosity of LOH). Recently, this mapping process has been revolutionized with the advent of microsatellite markers (small DNA repeat units) that are highly polymorphic and occur throughout the human genome. For example, one can test critical tumor suppressor gene loci on two chromosomal arms. One of the regions is on chromosomal arm 9p and contains the critical tumor suppressor gene called p16 (MTS-1 or CDKN2). This critical cell cycle inhibitor is inactivated by point mutation, deletion and methylation in most primary cancers. The second analysis can be done in chromosomal region 3p21, among the most frequently deleted regions in smoking related tumors. Although many genes have been isolated from the 3p region, none of the candidate genes have been inactivated frequently in primary tumors or cell lines. Importantly, both 9p and 3p losses are the earliest genetic changes identified in invasive cancer progression.

Patients with head and neck cancer often present with second primary tumors of the aerodigestive tract. Fundamental genetic studies in head and neck cancer show that single transformed cells in the oral cavity can spread throughout the mucosa giving rise to large areas of clonally related and transformed cells. There is further evidence that, when two lesions arise in one patient, they are often clonal in origin. For example, patients with head and neck cancer often have large patches of abnormal cells which can be directly tested by a simple biopsy.

See, e.g., Califano et al., Genetic Progression Model for Head and Neck Cancer. Cancer Research 56(11):2488-2492 (1996); Sidransky, Nucleic Acid-based Methods for Detection of Cancer. Science 278 (5340):1045-058 (1997); Rosin et al., Use of Allelic Loss to Predict Malignant Risk for Low-grade Oral Epithelial Dysplasia, Clinical Cancer Research 6:357362 (2000).

It being known, then, that an early prognosis that invasive cancer will eventually develop can be effected by genetic analysis of tissue from suspect sites, it would be highly desirable to provide a simple clinical protocol that would identify the locations of such suspect sites, well before onset of otherwise visible indications.

The Prior Art Mashberg Protocol

An in vivo diagnostic screening test is known which identifies and delineates suspect sites on epithelial tissue. This screening test, employing toluidine blue O (TBO) dye to selectively stain cancerous and precancerous tissue, is generally described in the U.S. Pat. No. 4,321,251 to Mashberg and in the U.S. Pat. No. 5,372,801 to Tucci et al. More recently kits have been developed which make it possible for clinicians to quickly and easily administer the test, as part of other routine dental or medical procedures, and thus identify and/or delineate suspect sites at a time when the patients are symptomless or while the dysplastic lesions are so small that they might be missed during normal visual examination. Once a suspect dysplastic lesion is identified by the Mashberg protocol, a regular biopsy sample was taken and subjected to conventional histological examination, to determine whether the lesion is malignant or precancerous. Kits for performing this test, containing premixed dye and rinse solutions in the proper quantities and concentrations, are licensed by Zila, Inc. and are available commercially in Canada, Australia and Europe under the trademark OraTest™. Later it was determined that other cationic dyes are similarly useful (See, e.g., U.S. Pat. No. 5,882,672 to Pomerantz) and that the selective marking action of such dyes is due to their uptake and retention by the mitochondria of cancerous and precancerous cells.

### EXAMPLE 1

### Clinical Testing Protocol

This example illustrates the conventional practice of the Mashberg protocol.

Preparation of Clinical Test Solutions

TBO (e.g., prepared in accordance with Example 1 of my U.S. Pat. No. 6,086,852), raspberry flavoring agent (IFF Raspberry IC563457), sodium acetate trihydrate buffering agent and H₂O₂ (30% USP) preservative (see U.S. Pat. No. 5,372,801), are dissolved in purified water (USP), glacial acetic acid and SD 18 ethyl alcohol, to produce a TBO test solution, having the composition indicated in Table A:

TABLE A

| Component | Weight % |
|---|---|
| TBO | 1.00 |
| Flavor | .20 |
| Buffering Agent | 2.45 |
| Preservative | .41 |
| Acetic Acid | 4.61 |
| Ethyl Alcohol | 7.48 |
| Water | 83.85 |
| | 100.00 |

Pre-rinse and post-rinse test solutions of 1 wt. % acetic acid in purified water, sodium benzoate preservative and raspberry flavor are prepared.

Clinical Protocol

The patient is draped with a bib to protect clothing. Expectoration is expected, so the patient is provided with a 10-oz. cup, which can be disposed of in an infectious waste container or the contents of which can be poured directly into the center of a sink drain, to avoid staining the sink. Environmental surfaces or objects which might be stained are draped or removed from the test area.

A visual oral cancer examination is conducted, without using any instruments which might cause nicks or cuts of soft tissues. Notations are made of the pre-staining appearance of soft tissues and teeth.

The patient rinses the oral cavity with approximately 15 ml. of the pre-rinse solution for approximately 20 seconds and expectorates, to remove excess saliva and provide a consistent oral environment. This step is then repeated with additional pre-rinse solution.

The patient then rinses and gargles with water for 20 seconds and expectorates.

The patient then rinses and gargles with 30 ml. of the TBO test solution for one minute and expectorates.

The patient then rinses with 15 ml. of the post-rinse solution for 20 seconds and expectorates. This step is then repeated.

The patient then rinses and gargles with water for 20 seconds and expectorates. This step is then repeated.

Observations of the oral cavity are then made, using appropriate soft-tissue examination techniques, including retraction, well-balanced lighting and magnification, if necessary. The location, size, morphology, color and surface characteristics of suspect lesions, that have retained blue coloration are made and recorded.

In order to reduce false positives, the patient is brought back after 10-14 days for a repeat of the above protocol. This period allows time for healing of any ulcerative or traumatic lesion or irritating etiology at the time of the first examination. A positive stain after the second examination of a suspect area detected in the first examination is considered an indication of cancerous or precancerous tissue and a biopsy is made to confirm this conclusion.

Early erythroplastic lesions stain blue, often in a stippled or patchy pattern. However, it normal for the stain to be retained by the irregular papiliar crevices on the dorsum of the tongue, which is not a positive indication. Other areas which retain blue stain, but are not regarded as positive include dental plaque, gingival margins of each tooth, diffuse stain of the soft palate because of dye transferred from the retained stain on the dorsum of the tongue, and ulcerative lesions which are easily distinguished. In all instances, where a lesion is highly suspect, but does not stain positively with this test, it is nevertheless imperative that a biopsy be taken.

“False Positives”

Until now it was understood that the Mashberg protocol could provide “false positive” indications, i.e., indications of cancerous or precancerous conditions which later conventional

histological examinations did not confirm, and considerable effort was expended to reduce the frequency of these false positives, e.g., by the repeated procedures described in the Mashberg patent and by special formulation of the dye as described in the Tucci patent.

Brief Statement of the Invention

However, it has now been determined clonal genetic changes indicative of eventual development of invasive cancer can be identified in suspected lesions that are stained by TBO and other cationic supravital dyes, even though conventional histological examination of such stained tissues do not confirm that the tissue is cancerous or precancerous. Thus, indications that were previously considered “false positives” in the Mashberg protocol are, in a high proportion of instances, the earliest indications of genetic alterations which are precursors to the development of invasive cancer. Thus, the Mashberg-type staining dye protocols are a reasonably reliable way to determine what tissues should be subjected to genetic analysis.

Accordingly, in considered in its broadest aspects I provide a prognostic method for early prediction of eventual development of invasive cancer, which combines the teachings of the prior art genetic analysis technology and the prior art selective staining dye technology, exemplified by the Mashberg protocol. My method comprises applying to tissue a staining dye that is selectively retained by mitochondria of preneoplastic cells, identifying clonal patches by visually examining said tissue for stained tissue sites, resecting tissue in the locus of said clonal patches and determining whether DNA extracted from said resected tissue exhibits allelic losses or mutation of tumor suppressor genes.

The following examples are provided to illustrate to those skilled in the art the practice of my invention and the presently preferred embodiments thereof. These examples are not to be understood as limiting the scope of the invention, which is defined only in the appended claims.

### EXAMPLE 1

### Mashberg-Type Clinical Protocol

Preparation of Clinical Test Solutions

TBO (e.g., the product of Example 1 of U.S. Pat. No. 6,086,852), raspberry flavoring agent (IFF Raspberry IC563457), sodium acetate trihydrate buffering agent and H₂O₂ (30% USP) preservative (See U.S. Pat. No. 5,372,801), are dissolved in purified water (USP), glacial acetic acid and SD 18 ethyl alcohol, to produce a TBO test solution, having the composition indicated in Table A:

TABLE A

| Component | Weight % |
|---|---|
| TBO Product | 1.00 |
| Flavor | .20 |
| Buffering Agent | 2.45 |
| Preservative | .41 |
| Acetic Acid | 4.61 |
| Ethyl Alcohol | 7.48 |
| Water | 83.85 |
| | 100.00 |

Pre-rinse and post-rinse test solutions of 1 wt. % acetic acid in purified water, sodium benzoate preservative and raspberry flavor are prepared.

Clinical Protocol

The patient is draped with a bib to protect clothing. Expectoration is expected, so the patient is provided with a 10-oz. cup, which can be disposed of in an infectious waste container or the contents of which can be poured directly into the center of a sink drain, to avoid staining the sink. Environmental surfaces or objects which might be stained are draped or removed from the test area.

A visual oral cancer examination is conducted, without using any instruments which might cause nicks or cuts of soft tissues. Notations are made of the pre-staining appearance of soft tissues and teeth.

The patient rinses the oral cavity with approximately 15 ml. of the pre-rinse solution for approximately 20 seconds and expectorates, to remove excess saliva and provide a consistent oral environment. This step is then repeated with additional pre-rinse solution.

The patient then rinses and gargles with water for 20 seconds and expectorates.

The patient then rinses and gargles with 30 ml. of the TBO test solution for one minute and expectorates.

The patient then rinses with 15 ml. of the post-rinse solution for 20 seconds and expectorates. This step is then repeated.

The patient then rinses and gargles with water for 20 seconds and expectorates. This step is then repeated.

Observations of the oral cavity are then made, using appropriate soft-tissue examination techniques, including retraction, well-balanced lighting and magnification, if necessary. The location, size, morphology, color and surface characteristics of suspect lesions, that have retained blue coloration are made and recorded.

The patient is brought back after 10-14 days for a repeat of the above protocol. This period allows time for healing of any ulcerative or traumatic lesion or irritating etiology that was present at the time of the first examination. A positive stain after the second examination of a suspect area detected in the first examination is considered an indication of cancerous or precancerous tissue.

Early erythroplastic lesions stain blue, often in a stippled or patchy pattern. However, it normal for the stain to be retained by the irregular papiliar crevices on the dorsum of the tongue, which is not a positive indication. Other areas which retain blue stain, but are not regarded as positive include dental plaque, gingival margins of each tooth, diffuse stain of the soft palate because of dye transferred from the retained stain on the dorsum of the tongue, and ulcerative lesions which are easily distinguished. In all instances, however, where a lesion is highly suspect, but does not stain positively with this test, it is nevertheless imperative that a biopsy be taken and subjected to molecular analysis.

### Example 2

### Genetic Alteration Analysis

58 samples from various clinical sites are obtained. It is determined that analysis of two of these samples is not possible because there is inadequate material on the slides for further molecular analysis. In the remaining 56 cases neoplastic cells are carefully dissected (in cases with cancer) from normal tissue or epithelium (in all other cases) from normal tissue using a laser capture microdissection scope. This allows isolation of the cells and extraction of DNA for subsequent microsatellite analysis at three critical loci. In 15 cases, there is insufficient DNA and further analysis is not possible. Two of the loci (D9S171 and D9S736) chosen for testing are on chromosomal region 9p21 which contains the p16 gene. A third marker (D3S1067) is located on chromosome 3p21. All molecular studies in the remaining 41 cases are done blinded without knowledge of the pathologic diagnosis.

Within the study, lesions that are stained blue and lesions that are biopsied adjacent to but not within the blue staining areas are separately identified. Thus, in many cases one is able to test both directly the stained areas as well as adjacent nonstained areas.

Microsatellite analysis of these critical markers in all of these 41 cases shows the presence of LOH (chromosomal deletions) in virtually all the cases with cancer and carcinoma in situ. In addition, many of the dysplastic lesions and nondysplastic lesions as well as those in the unknown (no pathologic diagnosis) category also harbor clonal genetic changes.

In 12 out of 12 cancer cases a clonal genetic change as expected is identified. In all four cases of carcinoma in situ or severe dysplasia a clonal change is also identified. In 57% of cases of dysplasia (4 out of 7) and 85% of cases without dysplasia (12 out of 14) clonal genetic changes are found in one or more of these markers. In cases with unknown histology clonal genetic changes are identified in 25% (1 of out 4) of the cases. Overall, clonal changes are identified by microsatellite analysis in 80% of the lesions (33 out of 41). This molecular analysis definitively shows that approximately 80% of the lesions identified by the Mashberg-type protocol are clonal.

### CONCLUSIONS

Previous studies suggest that only lesions with clonal genetic changes are likely to progress to cancer. All of these studies suggest that only lesions with 9p and/or 3p loss will proceed to cancer, lesions without these changes virtually never progressed. The risk of progression in preneoplastic lesions clearly rises to virtually 100% if other more advanced genetic changes (e.g., 17p loss or p53 mutation) are present.

The risk of progression to cancer with 3p and/or 9p loss varies from 28-75%. However, in the study with the lowest frequency of progression (28%), the 116 patients prospectively tested were free of cancer prior to entering the study. The other two studies included 123 patients with a previously resected primary tumor and reflected a higher rate or progression (45-78%). In the most recent study, the authors made a clear recommendation to resect all suspicious areas with loss of two or more alleles.

This Example thus establishes the fact that preneoplastic changes identified by the toluidine blue dye in this patient population are clonal and are therefore in the progression pathway to cancer. Although it is not certain that every one of the identified lesions will progress to an invasive cancer it is clear that the initial clonal expansion has begun in these patients. Based on the above mentioned studies a conservation estimate would suggest that 50-75% of these lesions are likely to progress to invasive cancer. Therefore, these clonal patches place these patients in a very high risk category and that complete excision of the affected area is warranted.

Another important point derived from this Example relates to the issue of staining and nonstaining areas. In almost all cases both the staining and nonstaining areas share the same clonal genetic changes. Based on elucidation of the clonal progression model of head and neck cancer, it is clear that most patients with head and neck cancer that recur have large patches of abnormal epithelium. It is not surprising that a biopsy away from the staining area would also show the same clonal genetic changes. Importantly, there are at least three cases where only the biopsy from the stained region revealed a clonal genetic change while the unstained areas did not show these changes. It is probably due to the presence of smaller clonal patches in these patients and further supports the use of dye staining in identifying abnormal lesions.

Clearly, the Mashberg-type protocol identifies patients with carcinoma in situ and cancer confirmed by standard morphologic analysis. Remarkably, the vast majority of other deleted lesions appeared normal or dysplastic under the microscope (and would have otherwise been classed as a “false positive” from the Mashberg protocol, but still those lesions harbor the critical clonal genetic changes that are necessary for cancer progression.

Thus, the Mashberg-type protocol represents a powerful method to detect cancers as well as lesions that are very likely to progress to cancer. In this regard, it is appropriate to biopsy the lesions identified by staining, which may (A) result in a cure because the lesion can be removed in it entirety by further resection, or (B) appropriately place patients in a high risk category by documenting clonal genetic progression.

Having disclosed my invention in such terms as to enable those skilled in the art to understand and practice it, and, having identified the presently preferred embodiments thereof, I claim:

1. A prognostic method for early prediction of eventual development of invasive cancer, said method comprising:
(a) applying to tissue a staining dye that is selectively retained by mitochondria of neoplastic and preneoplastic cells;
(b) identifying clonal patches of said tissue by visually examining said tissue for stained tissue sites;
(c) resecting tissue in the locus of said clonal patches;
(d) extracting DNA from said resected tissue; and
(e) determining whether DNA extracted from said resected tissue exhibits allelic losses or mutation of tumor suppressor genes.
